(19) United States
(12) Patent Application Publication
(10) Pub. No.: US 2005/0113875 A1
(43) Pub. Date: May 26, 2005

(54) Dynamic blanking and recharge intervals for cardiac rhythm management

(76) Inventor: David Ternes, Roseville, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/723,023

(22) Filed: Nov. 26, 2003

(51) Int. Cl. A61N 1/362

(52) U.S. Cl. 607/9

(57) ABSTRACT

A cardiac rhythm management device in which sensing amplifiers are blanked for a dynamically adjusted recharge interval after a pacing pulse is delivered by a pacing channel. Recharge intervals are dynamically adjusted in accordance with measured and/or programmable parameters that affect the optimum recharge time in order to reduce the total time in which sensing is disabled during a cardiac cycle.

### FIELD OF THE INVENTION

This invention pertains to cardiac rhythm management devices such as pacemakers and implantable cardioverter/defibrillators.

### BACKGROUND

Cardiac pacemakers are implantable medical devices that provide electrical stimulation in the form of pacing pulses to selected chambers of the heart (i.e., the atrium and/or ventricle). As the term is used herein, a pacemaker is any cardiac rhythm management device that performs cardiac pacing, including implantable cardioverter/defibrillators having a pacing functionality. In order to cause a cardiac contraction in the absence of an intrinsic beat, a pacing pulse (either an atrial pace or a ventricular pace) with energy above a certain pacing threshold is delivered to a heart chamber by an electrode in electrical contact with the myocardium. A wave of depolarizing excitation then propagates through the myocardium, resulting in a heartbeat.

Pacemakers typically have a programmable electronic controller that causes the pacing pulses to be output in response to lapsed time intervals and sensed electrical activity (i.e., intrinsic heart beats not as a result of a pacing pulse). The manner in which pacing pulses are output is defined by a pacing mode, and modem pacemakers are programmable to operate in a number of different modes. Pacemakers are most often programmed to operate in some sort of demand type pacing mode where a pacing pulse is delivered to a heart chamber unless intrinsic activity is sensed in the chamber before expiration of an escape interval. The device senses intrinsic cardiac electrical activity by means of internal electrodes disposed near the chamber to be sensed. A depolarization wave associated with an intrinsic contraction of the atria or ventricles that is detected by the pacemaker is referred to as an atrial sense or ventricular sense, respectively. When a pacing pulse is output by the device, sensing of intrinsic activity is temporarily disabled for a period of time referred to as a blanking interval in order to prevent the pacing pulse from saturating the sensing amplifiers. The blanking interval also extends for some time after the pacing pulse in order to allow afterpotentials at the pacing electrode to dissipate. Outputting of a recharging pulse after each pacing pulse can be used to both recharge the pulse output circuit and to actively dissipate such afterpotentials in a shorter period of time. The sensing amplifiers are then blanked for the duration of all pacing and recharging pulses during a cardiac cycle.

### SUMMARY OF THE INVENTION

During a blanking interval, a pacemaker is blinded to all intrinsic cardiac activity. This may adversely affect pacemaker operation if device fails to sense intrinsic beats that would otherwise inhibit paces or fails to detect tachyarrhythmias for which appropriate therapy should be delivered. The present invention is directed toward reducing the time during which sensing amplifiers are blanked in a cardiac rhythm management device. In one embodiment, the duration of recharging pulses for a pacing channel are dynamically adjusted based upon measured or programmed parameters that affect the time required to recharge the pacing channel. Such dynamic adjustment allows shorter recharging intervals than if fixed nominal recharging intervals are used and also allows a shorter corresponding blanking interval for the sensing amplifiers.

### BRIEF DESCRIPTION OF THE DRAWINGS

FIG. 1 is a block diagram of a multi-site cardiac rhythm management device.

FIG. 2 is a schematic of a basic pulse output circuit.

FIG. 3 is a timing diagram showing an exemplary sequence of pacing and recharging pulses.

### DETAILED DESCRIPTION

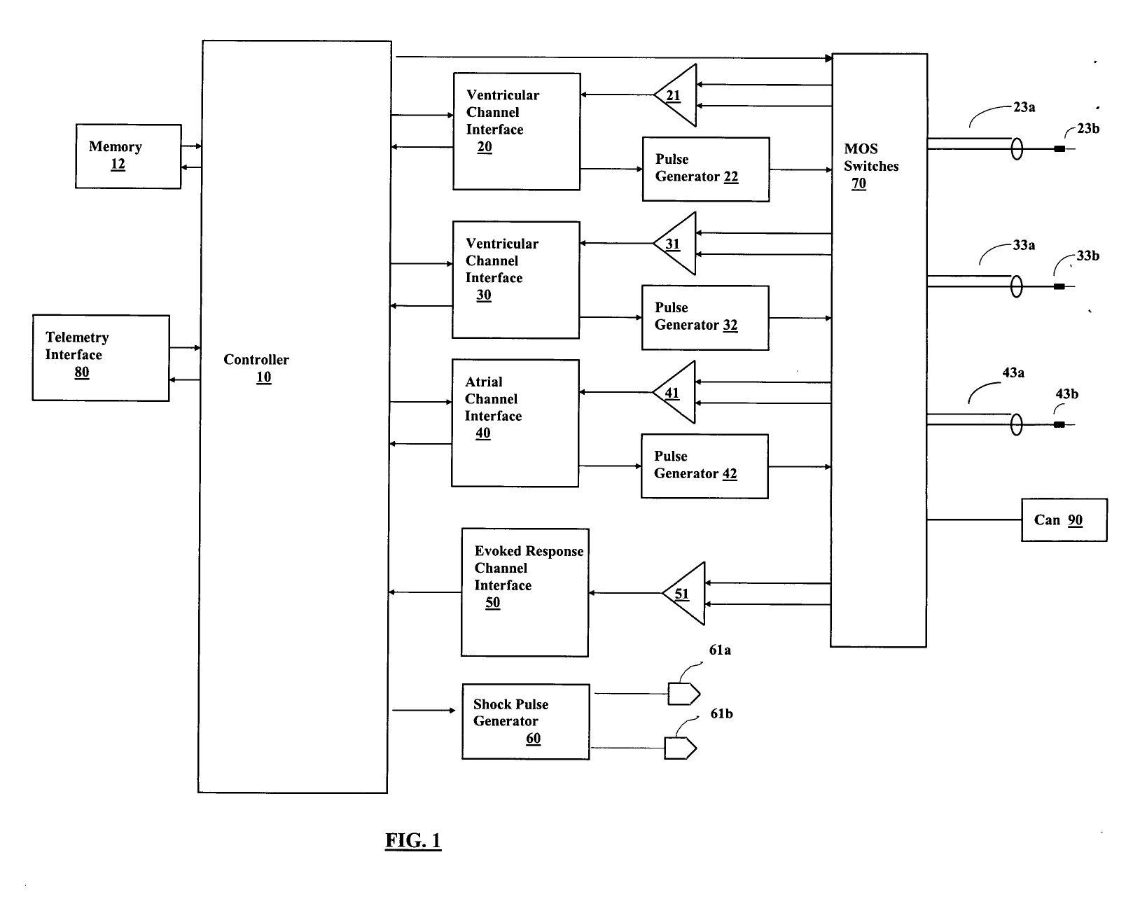

A block diagram of cardiac rhythm management device with cardioversion/defibrillation capability and having an atrial and two ventricular pacing channels is shown in FIG. 1. The controller of the device is made up of a microprocessor 10 communicating with a memory 12 via a bidirectional data bus 13, where the memory 12 typically comprises a ROM (read-only memory) for program storage and a RAM (random-access memory) for data storage. The controller could be implemented by other types of logic circuitry (e.g., discrete components or programmable logic arrays) using a state machine type of design, but a microprocessor-based system is preferable. The controller is capable of operating the device in a number of programmed modes where a programmed mode defines how pacing pulses are output in response to sensed events and expiration of time intervals. A telemetry interface 80 is also provided for communicating with an external programmer.

The pacemaker has an atrial sensing/pacing channel comprising ring electrode 43a, tip electrode 43b, sense amplifier 41, pulse generator 42, and an atrial channel interface 40 which communicates bidirectionally with a port of microprocessor 10. The device also has two ventricular sensing/pacing channels that similarly include ring electrodes 23a and 33b, tip electrodes 23b and 33b, sense amplifiers 21 and 31, pulse generators 22 and 32, and ventricular channel interfaces 20 and 30. The electrodes are electrically connected to the device by means of a lead (not shown) and to the pulse generators and sense amplifiers through a MOS switching circuit 70. The switching circuit 70 enables the inputs of a sense amplifier or outputs of a pulse generator to be selectively connected to either both electrodes of the sensing/pacing channel or to only one of the electrodes with the other input or output electrically connected to the device housing, designated as the can 90 in the figure. In this way, either bipolar or unipolar sensing/pacing can be enabled. For each channel, the same lead and electrode are normally used for both sensing and pacing. The pacemaker also has an evoked response sensing channel that comprises an evoked response channel interface 50 and a sense amplifier 51 and that may be connected to any of the sensing channel electrodes by means of the switching circuit 70. Sensing of an evoked response to a pacing pulse allows the device to verify whether or not a pacing pulse has captured the heart and make adjustments to the duration and/or magnitude of subsequent paces. The channel interfaces include analog-to-digital converters for digitizing sensing signal inputs from the sensing amplifiers, registers that can be written to for adjusting the gain and threshold values of the sensing amplifiers, and, in the case of the ventricular and atrial channel interfaces, registers for controlling the output of pacing pulses and/or changing the pacing pulse amplitude or duration. A shock pulse generator 60 is also provided for delivering defibrillation shocks through shock electrodes 61a and 61b.

During the time that a pacing pulse is output through a sensing/pacing channel, sensing amplifiers are blanked (i.e., disabled) in order to prevent saturation of the sensing amplifiers and to prevent crosstalk between channels where a pace to one electrode site is interpreted as an intrinsic depolarization at another electrode site. Blanking of a sensing amplifier may be implemented in the switching network 70 by, for example, disconnecting the amplifier inputs from the electrode or short circuiting the amplifier inputs to ground. Blanking may alternatively be implemented by disconnecting the power source from a sensing amplifier. By whatever means blanking is accomplished, when a pacing pulse (or defibrillation shock) is delivered to the heart, the controller operates the switching network so as to blank the sensing amplifiers of all the sensing/pacing channels for a defined time period, referred to as the blanking interval. It is common practice for blanking intervals in cardiac pacemakers to not only last for the duration of the pacing pulse, but to also extend beyond the end of the pulse. The reason for this is that, after a pulse is output from an electrode, there is a residual post-stimulus potential or afterpotential that arises from stored charges at the electrode/electrolyte interface. These afterpotentials are much larger than the potentials that arise from an intrinsic heartbeat and can be sensed not only by the electrode from which the pulse was delivered, but by electrodes located at other cardiac sites. In order to prevent false sensing by the sensing channels, therefore, the blanking interval should be made long enough so that afterpotentials have time to dissipate.

The time required for a pacing electrode to recover can be shortened by actively dissipating the afterpotential. FIG. 2 illustrates one method for doing this that concomitantly recharges the pulse output circuit. FIG. 2 is a circuit diagram illustrating a pulse output circuit 100 that is representative of the pulse generators 22, 32, or 42 in FIG. 1. In order to provide pulses of sufficient amplitude and duration, a capacitive discharge circuit is used. The pulse output circuit 100 delivers pacing pulses to the heart of a patient under the direction of the microprocessor-based controller by performing a charging cycle, a pacing cycle, and a recharging cycle. The circuit is made up of two transistors TR1 and TR2 and an output capacitor C1 that is connected to a load resistance R1 made up of the heart and an electrode of a pacing channel. In this example, the circuit delivers a unipolar pace by discharging the voltage across the output capacitor between the electrode and the device housing, designated as ground in the figure. In bipolar pacing, the capacitor voltage would be discharged across the tip and ring electrodes of a sensing/pacing channel. The conducting states of the transistors are determined by their base voltages as controlled by a switching circuit SC1 in accordance with control signals CS from the microprocessor. The output capacitor C1 is charged to the supply voltage Vₛ when transistor TR1 conducts while transistor TR2 is turned off. In order to deliver a pacing pulse, transistor TR1 is turned off while transistor TR2 is activated, pulling the collector of TR2 to ground and causing the charged capacitor to discharge across the load resistance. The supply voltage Vₛ is normally made positive so that the heart is paced with a negative or cathodal pacing pulse. After the pacing pulse is delivered, transistor TR1 is turned on to recharge the capacitor. Recharging of the capacitor causes a voltage pulse with positive polarity to appear across the load resistance, but the pulse is delivered during the refractory or non-excitable period of the heart so no anodal stimulation results. Besides recharging the capacitor C1, the recharge pulse also actively dissipates afterpotentials on the pacing electrode and shortens the time that the sensing amplifiers must be blanked for reliable sensing. In order to prevent their saturation, sensing amplifiers are blanked not only during output of pacing pulses, but also during any recharge pulses.

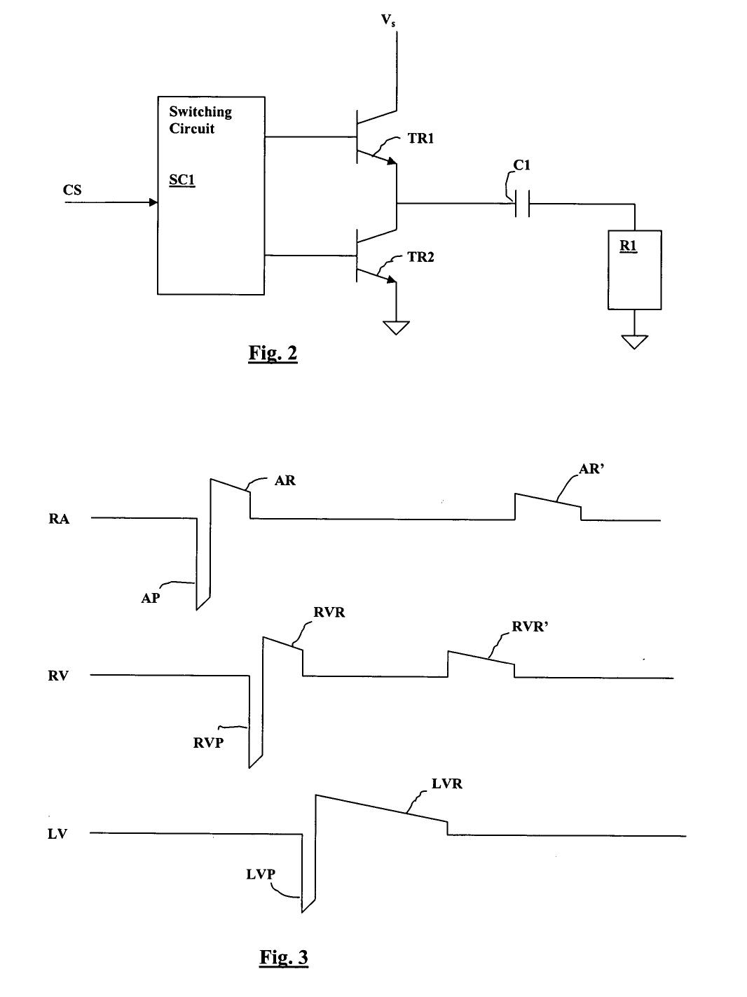

In devices such as depicted in FIG. 1 that are capable of delivering multiple pacing pulses to different heart chambers or pacing sites, all of the sensing amplifiers are blanked while any pacing channel is either delivering a pacing pulse or being recharged. Since multiple paces may be delivered during a cardiac cycle, a recharge cycle for one pacing channel may be interrupted until a pacing and recharge cycle is completed for another pacing channel, at which point the interrupted recharge cycle is restarted. For example, the device of FIG. 1 may be configured to deliver atrial and biventricular pacing where the ventricular paces are separated by a specified offset interval. FIG. 3 illustrates the pacing and recharge pulses output during an exemplary cardiac cycle where the recharging pulse for each channel is set to a nominal value of 30 milliseconds. Timelines for the right atrial, right ventricular, and left ventricular pacing channels are labeled RA, RV, and LV, respectively. An atrial pace is followed 20 ms later with a bi-ventricular pace having a specified offset interval such that a right ventricular pace is delivered 10 milliseconds before the left ventricular pace. The atrial pace AP is thus delivered and 20 ms of atrial recharge AR occurs before the right ventricular pace RVP is delivered. Then, 10 milliseconds of right ventricular recharge RVR occurs before the left ventricular pace LVP is delivered. After, the left ventricular pace is delivered, the 30 millisecond left ventricular recharge LVR starts and completes, followed by the remaining 20 ms of right ventricular recharge RVR′ and finally the last 10 ms of atrial recharge AR′. A similar sequence of events occurs in other multi-site pacing situations where paces are delivered to both atria or to multiple sites in a single heart chamber.

One way that the sequence of interrupted and restarted recharging cycles illustrated in FIG. 3 may be accomplished is by the use of a stack architecture in the controller programming that restarts the recharging of each pacing channel on a “last in, first out” basis. Thus, when the right ventricular pace RVP occurs before the right atrial recharge AR has completed, the right ventricular pace interrupts the recharge in the atrial channel and “pushes” that channel onto the stack. After that, a left ventricular pace occurs before the right ventricular recharge RVR has completed, and the left ventricular pace interrupts the recharge in the right ventricular channel and “pushes” that channel onto the stack. When the left ventricular recharge is complete, the stack is “popped”, and the right ventricular recharge is restarted. Finally, the right ventricular recharge completes, the stack is “popped” again, and the atrial recharge is completed.

The example of FIG. 3 shows that the recharge times for all of the pacing channels in a multi-site pacemaker are cumulative. As the sensing amplifiers are blanked during the entire time interval when either pacing pulses are being output or a recharging cycle for a pacing channel has yet to be completed, the device is blind to intrinsic cardiac activity for a significant period of time. This potentially impairs detection of tachyarrhythmias and results in long gaps in a recording of the sense signals generated by the sensing channels (i.e., an electrogram) as the A/D samples are flat-lined during the blanking interval. It may also adversely affect pacing and interfere with sensing of evoked potentials in order to verify capture by pacing pulses.

The total time in which the sensing amplifiers must be blanked can be reduced by providing a recharge interval for each pacing channel that is dynamically adjusted in accordance with one or more measured or programmed parameters, either in addition to or instead of the pulse amplitude. As aforesaid, the amplitude of the pacing pulse has been used in previous devices to adjust the recharge interval so that the recharge interval increases as the pulse amplitude increases. Other parameters may also be used to adjust the optimum recharge interval, however, including the measured voltage droop during a pacing pulse, pacing pulse duration, measured lead impedance, programmed AV delay between atrial and ventricular paces, and programmed offset interval between ventricular paces in biventricular paces. Optimum recharge intervals can be determined empirically by testing an individual device with different programmed or measured parameters. In one embodiment, such determination of optimum recharge intervals may be made by an external programmer based upon inputs received from the implanted device, where the optimum recharge intervals are determined by calculation or through the use of a look-up table having empirically determined optimum recharge intervals for different parameters. In another embodiment, one or more measurable pacing or programmable pacing parameters along with corresponding optimum recharge intervals based upon different values for these parameters are incorporated into a look-up table implemented in the controller's programming. The controller's programming is then able to use the look-up table to dynamically adjust the duration of the recharge interval for each pacing channel as parameters change rather using a fixed nominal value.

In another embodiment, rather than determining optimum recharge intervals with a look-up table generated by empiric testing of the device, a formula can be used that allows optimum recharge intervals to be directly calculated based upon measurements and/or programmable settings of selected parameters. Such optimum recharge intervals can then either be calculated as they are needed by the device or periodically calculated and stored in a look-up table. An exemplary such formula for calculating an optimum recharge interval T_{recharge} is as follows:
T_{recharge}=−RC₁(ln(2V_{droop}/Vᵢ/(1−e^{PW/RC})))
Where R is the measured lead impedance, C₁ is the measured lead capacitance, V_{droop} is the measured voltage droop during a pacing pulse, Vᵢ is the pacing pulse amplitude, PW is the pacing pulse duration, and C is the total measured capacitance. Such updating of the optimum recharge interval may be performed at specified time intervals or on a beat-to-beat basis.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

1. A cardiac rhythm management device, comprising:
one or more sensing channels for sensing depolarizations in a heart chamber and generating sense signals in accordance therewith, each such sensing channel including a sensing amplifier that can be connected to an electrode;
one or more pacing channels for delivering pacing pulses to one or more selected pacing sites;
a controller for controlling the delivery of pacing pulses in accordance with sensing signals and elapsed time intervals;
wherein the controller is programmed to recharge a pacing channel following a pacing pulse by outputting a recharging pulse for a specified recharging interval and to blank the sensing amplifiers during the time a pacing or recharging pulse is output; and,
wherein the controller is further programmed to dynamically adjust the specified recharging interval based upon a measured parameter.

2. The device of claim 1 wherein the controller is programmed to dynamically adjust the specified recharging interval based upon a programmed pacing pulse amplitude setting.

3. The device of claim 1 wherein the controller is programmed to dynamically adjust the specified recharging interval based upon a programmed pacing pulse duration setting.

4. The device of claim 1 wherein the controller is programmed to dynamically adjust the specified recharging interval based upon a programmed AV interval between an atrial and a ventricular pacing pulse.

5. The device of claim 1 wherein the controller is programmed to dynamically adjust the specified recharging interval based upon a programmed offset interval between ventricular paces during biventricular pacing

6. The device of claim 1 wherein the controller is programmed to dynamically adjust the specified recharging interval based upon a measured lead impedance.

7. The device of claim 1 wherein the controller is programmed to dynamically adjust the specified recharging interval based upon a measured voltage droop during a pacing pulse.

8. The device of claim 1 wherein the controller is programmed to dynamically adjust the specified recharging interval T_{recharge} based upon the following formula:
T_{recharge}=−RC₁(ln(2V_{droop}/Vᵢ/(1−e^{PW/RC})))
where R is a measured lead impedance, C₁ is a measured lead capacitance, V_{droop} is a measured voltage droop during a pacing pulse, Vⁱ is a programmed pacing pulse amplitude, PW is a programmed pacing pulse duration, and C is a total measured capacitance.

9. The device of claim 1 wherein the controller is programmed to dynamically adjust the specified recharging interval by using a look-up table that contains optimum recharge intervals corresponding to one or more programmable or measured pacing parameter values.

10. The device of claim 9 wherein the optimum recharge intervals corresponding to various parameter values are determined empirically by device testing.

11. A method for operating a cardiac rhythm management device, comprising:
sensing depolarizations in a heart chamber through one or more sensing channels and generating sense signals in accordance therewith, each such sensing channel including a sensing amplifier that can be connected to an electrode;
delivering pacing pulses through one or more pacing channels in accordance with a programmed pacing mode;
recharging a pacing channel following a pacing pulse by outputting a recharging pulse for a specified recharging interval and blanking the sensing amplifiers during the time a pacing or recharging pulse is output; and,
dynamically adjusting the specified recharging interval based upon a measured parameter.

12. The method of claim 11 further comprising dynamically adjusting the specified recharging interval based upon a programmed pacing pulse amplitude setting.

13. The method of claim 11 further comprising dynamically adjusting the specified recharging interval based upon a programmed pacing pulse duration setting.

14. The method of claim 11 further comprising dynamically adjusting the specified recharging interval based upon a programmed AV interval between an atrial and a ventricular pacing pulse.

15. The method of claim 11 further comprising dynamically adjusting the specified recharging interval based upon a programmed offset interval between ventricular paces during biventricular pacing.

16. The method of claim 11 further comprising dynamically adjusting the specified recharging interval based upon a measured lead impedance.

17. The method of claim 11 further comprising dynamically adjusting the specified recharging interval based upon a measured voltage droop during a pacing pulse.

18. The method of claim 11 further comprising dynamically adjusting the specified recharging interval T_{recharge} based upon the following formula:
T_{recharge}=−RC₁(ln(2V_{droop}/Vᵢ/(1−e^{PW/RC})))
where R is a measured lead impedance, C₁ is a measured lead capacitance, V_{droop} is a measured voltage droop during a pacing pulse, Vᵢ is a programmed pacing pulse amplitude, PW is a programmed pacing pulse duration, and C is a total measured capacitance.

19. The method of claim 11 further comprising dynamically adjusting the specified recharging interval by using a look-up table that contains optimum recharge intervals corresponding to one or more programmable or measured pacing parameter values.

20. The method of claim 19 wherein the optimum recharge intervals corresponding to various parameter values are determined empirically by device testing.
